(19) United States
(12) Patent Application Publication
(10) Pub. No.: US 2025/0198901 A1
(43) Pub. Date: Jun. 19, 2025

(54) SYSTEM AND METHOD FOR DETERMINING THE FLUID FLOW WITH TRACER IN FRACTURED ROCK SAMPLE

(71) Applicant: Petróleo Brasileiro S.A. - Petrobras, Rio de Janeiro (BR)

(72) Inventors: Mario Germino Ferreira da Silva, Rio de Janeiro (BR); Flavia de Oliveira Lima Falcao, Rio de Janeiro (BR); Andre Gustavo di Palma Cordovil, Rio de Janeiro (BR)

(73) Assignee: Petróleo Brasileiro S.A. - Petrobras, Rio de Janeiro (BR)

(21) Appl. No.: 18/979,381

(22) Filed: Dec. 12, 2024

(30) Foreign Application Priority Data
Dec. 13, 2023 (BR) 1020230262716

(51) Int. Cl. G01N 15/08; G01N 23/04; G01N 33/24

(52) U.S. Cl. CPC G01N 15/0826; G01N 23/04; G01N 33/24; G01N 2223/616

(57) ABSTRACT

The present invention describes the development of a laboratory system for testing rock samples with fractures subjected to single-phase flow, using visible fluid and equipment made of material not sensitive to a microtomograph, allowing (i) the quantification of parameters that represent the flow in a fractured medium in the flow simulator and (ii) images that portray the distribution of fluids in the medium, in different injection steps.

### FIELD OF INVENTION

The present invention relates to the study of fluid flow through fractured rocks. More specifically, the present invention relates to a system for studying the influence of natural fractures, which are found in pre-salt reservoirs, on the fluid flow within the porous medium and for understanding the fluid flow in multi-fractured wells.

### BACKGROUND OF THE INVENTION

Every oil reservoir contains fractures, whether natural or induced, and on different scales. In general, natural fractures can be the result of two geological processes that occur after sedimentary deposition: tectonic efforts in the subsurface and/or dissolution by percolation of acidic fluid. On the other hand, so-called induced fractures are the result of activities in which human action changes the balance of tensions in which the rocky environment was initially found, such as drilling, hydraulic fracturing or the development of the field itself. While the first two are restricted to the region neighboring the wells, the last one can occur in any location of the reservoir, depending on the pore pressure, the compartmentalization of the reservoir or the environment heterogeneities.

Regardless of the fracture nature, whether natural or induced, they exert an important influence on the hydrocarbon flow in the porous medium (i.e. in oil reservoirs), and their behavior must be represented in the fluid flow simulator in the reservoir. This representation allows the model to be adjusted to production data, which gives greater robustness to the prediction of reservoir production.

The representation of fractures in the reservoir model requires parameters that describe their contribution to the fluid flow in the porous medium, such as the permeability of the fractures and the transfer function, which, as the name suggests, quantifies the transfer of fluids between the domains of the fracture and the porous medium. For a better representation of the physical phenomenon, i.e. the contribution of fractures to the fluid flow in the fractured porous medium, parameter values representative of the medium in question should be used. To this end, one possibility is to obtain experimental results from laboratory tests on samples of fractured rock, subjected to flow.

A second application of laboratory tests, in addition to obtaining parameters to represent the contribution of fractures to the flow in the fractured medium (which may be natural or induced fractures) in reservoir simulation, is their contribution to understanding the path taken by the fluid within the fractured medium. An example of the use of laboratory-scale observation at the field scale is the best positioning of production and injection wells based on the characteristics of the fractures, including quantity, dimensions, geometry and direction. For this, it is necessary that the flow experiment in a fractured porous medium be carried out inside the microtomograph, since the image with the distribution of the fluid in the sample allows such understanding. For an image acquisition, it is important that the percolating fluid be visible to the microtomograph, while the equipment used to wrap the sample is transparent. Therefore, the test must consist of different steps of fluid injection into the fractured rock sample; at the end of each one, an image will be obtained that will highlight the distribution of the fluids.

### PRIOR ART

Publication WO0150819A1 A reveals measurements of variations in fracture porosity and permeability with increasing effective stress of cylindrical rock core plugs of various types (carbonate, silicoclastic, shale, for example) and dimensions. The test can be implemented for natural and induced fractures propagating axially in the rock core. Testing begins with initial testing, sample preparation and obtaining measurements on an intact plug to define the matrix properties in the rock core plug. A rock core plug with defined matrix properties is then, after further preparation, subjected to an axial shear fracture (natural or induced) that propagates through its body. Measurements are then obtained from the plug with an axial shear fracture (natural or induced) propagating through its body to determine fracture properties.

More specifically, this prior art discloses, among others, a method for laboratory measurement of dynamic variations in the petrophysical properties of an underground formation as a result of changes in the stress applied to a core rock sample obtained from a location of interest in the underground formation. Computed tomography is performed on the core rock sample to determine the homogeneous density distribution in the rock sample. A measurement of a relationship between the sensitivity to matrix porosity and the sensitivity to matrix permeability of the rock sample is then determined. An axially extending shear fracture is propagated through the rock sample to form a split core plug composed of fracture halves of the rock sample. The fracture halves of the split core plug are axially displaced relative to each other to simulate displacement during in situ fracturing of the formation rock. The fracture-displaced halves of the split core plug are then joined to form a bonded split plug. A measure is then determined of the stress sensitivity of the fracture porosity and fracture permeability of the core rock sample (paragraphs 5 to 7).

In general terms, the method of this reference consists of selecting 108 a cylindrical plug representative of an area of interest and perform computed tomography (CT) tests on it to analyze the internal structure of the plug matrix and check whether discontinuities or high/low density inclusions are detected. Examples of CT include medical computed tomography and micro-CT. Conventional core analysis (CCA) 112 is also performed to determine plug characteristics (paragraphs 27-70).

Then the plug is subjected to intact plug stress sensitivity test 106 and pore volume compressibility (PVC) test 114. During the PVC test, a volume of water expelled by the sample under increasing confining pressure is measured. See paragraph 46. Other tests are also performed (116, 118). After this, the plug is fractured 122 by applying a load at a constant rate until an axial tensile fracture is created. In the next step 124, the two resulting fracture halves (faces) 220a and 220b (FIG. 12) are slid past each other manually or mechanically to simulate the displacement naturally experienced when a shear stress component acts on the rock during the in situ fracturing process (paragraphs 63-66).

Finally, tests similar to those performed for the intact plug are repeated for the fractured plug, namely: Computed tomography analysis 126, fracture porosity and permeability measurement 128, fracture stress sensitivity 130, and PVC+SDk testing 132 (paragraphs 70-82).

Patent application CN113029910A discloses a rock core holder used in cooperation with a rock seepage real-time imaging system and a rock core holder method, and relates to the field of rock seepage mechanics. The rock core holder is characterized in that an inlet plug, a pressure chamber and an outlet plug are connected sequentially from left to right to form an external structure; and in the external structure, the left end, the left infiltration joint, the rock sample, the right infiltration joint and the right end are connected sequentially from left to right to form an internal structure. The test method comprises the following steps: (1) sample preparation; (2) sample loading; (3) testing; and (4) recycling. The device can be matched with a rock seepage real-time imaging system, the confining pressure condition in the actual rock stratum is simulated by using the pressure of the confining pressure medium in the pressure chamber, and the permeability test of the rock sample under different stress conditions is carried out by using a steady state method or a transient state method to obtain the permeability stress sensitivity rule of the rock sample. The method is uniform standard, can realize multiple industrial replications, and is suitable for in-situ real-time permeability testing and imaging of various medium porous rocks or fractured rocks.

In other words, this reference is to use existing imaging technology and permeability testing technology to provide a core support and a method used in conjunction with a rock seepage real-time imaging system, so as to realize real-time imaging of rock samples in the process of rock permeability testing. Real-time acquisition of parameters such as infiltration channel, water saturation, water distribution or pore structure.

Core support includes rock samples, infiltration joints, ends, heat shrinkable tubes, elastics, inlet plugs, outlet plugs, pressure chambers, threads, infiltration inlet valves, infiltration outlet valves, booster valves, discharge pressure valve, exhaust valve, confining pressure medium and infiltration medium.

The reference can cooperate with the rock seepage real-time imaging system, use the pressure of the confining pressure medium in the pressure chamber to simulate the confining pressure conditions in the actual rock formation, and use the steady-state method or the transient method to realize the penetration of rock samples under different stress conditions. Rate test to obtain the permeability stress sensitivity law of rock sample. At the same time, the real-time imaging system can perform real-time imaging of the sample during the test, and realize dynamic acquisition of parameters such as infiltration channel, water saturation, water distribution or pore structure. D2 has uniform standards, can realize multiple industrial replications, and is suitable for permeability testing and real-time in-situ imaging of various porous media rocks or rocks containing fractures.

Rock sample 1 is a standard cylindrical sample of porous media or fractured rock with flat ends. The diameter and height of sample 1 meet the permeability test requirements (FIGS. 1 and 2).

### SUMMARY OF THE INVENTION

The present invention discloses a system and method of using the same device for testing rock samples with fractures subjected to single-phase flow, using tracer fluid and equipment invisible to a microtomograph. A porous plug is artificially fractured and wrapped with a liner comprising holes. The plug and liner are received in a receptacle in fluid communication with a pump. The pump flows the tracer fluid through the receptacle, forcing the fluid through the porous plug, fractures, and finally through the liner ports. The flow is recorded by a microtomograph. The flow data thus obtained allow (i) the quantification of parameters that represent the flow in a fractured medium in a flow simulator and (ii) images that show the distribution of fluids in the medium, at different injection steps.

### BRIEF DESCRIPTION OF THE DRAWINGS

The present invention will now be described below with reference to the typical embodiments thereof and also with reference to the attached drawings, in which:

FIG. 1 is a representation of an illustrative diagram of the fractures contribution, in terms of porosity and permeability of the total reservoir, compared with the rock matrix contribution in a fluid separator vessel coupled to the monitoring system, according to Nelson, 2001;

FIG. 2 is an oil recovery graph for water-wet (Iaw=1.0) and non-water-wet (Iaw=0.09) reservoirs according to Firoozabadi (2000);

FIG. 3 is a graph of increased suction (capillary pressure) for non-water-wettable reservoirs (Iaw=0.09) according to Firoozabadi (2000);

FIG. 4 is a representation of components of a naturally fractured reservoir according to Phelps and Strauss (2002);

FIG. 5 is a representation of an integrated methodology for the development of naturally fractured reservoirs, adapted from Bourbiaux et al. (2005);

FIG. 6 is a representation of the construction of a multi-scale fracture model (DFN model) according to Bourbiaux et al. (2005);

FIG. 7 is a representation of a porous plug according to the state of the art;

FIG. 8 is a representation of an artificially fractured porous plug in accordance with the present invention;

FIG. 9 illustrates alternative representations of artificially fractured porous plugs according to the present invention;

FIG. 10 is a representation of a viton liner comprising holes according to the present invention;

FIG. 11 is a representation of the viton liner surrounding the porous plug according to the present invention;

FIG. 12 is a representation of the system for determining fluid flow with tracer in a fractured rock sample according to the present invention;

FIG. 13 is a representation of adapters together with the viton liner and porous plug assembly according to the present invention;

FIG. 14 is a representation of the system according to the present invention located in a microtomograph;

FIG. 15 is a detailed representation of the adapters according to the present invention.

### DETAILED DESCRIPTION OF THE INVENTION

Specific embodiments of this disclosure are described below. In an effort to provide a concise description of these embodiments, all features of an actual implementation may not be described in the specification. It should be appreciated that in the development of any actual implementation, as in any engineering project or design, numerous implementation-specific decisions must be made to achieve developers' specific goals, such as compliance with system- and business-related constraints, which can vary from one implementation to another. Furthermore, it should be appreciated that such a development effort may be complex and time-consuming, but would nevertheless be a routine design and manufacturing undertaking for those of ordinary skill having the benefit of this disclosure.

### Basis of the Concepts of the Invention

In order to facilitate understanding of the result application to be obtained from the laboratory tests to be performed with the system of the present invention, some related definitions are presented below.

### I—Definition of a Naturally Fractured Reservoir

Below are highlighted some of the main geological factors related to the formation of naturally fractured reservoirs:
Lithology: most reservoir rocks are of sedimentary origin, the main ones being sandstones and carbonates. Natural fractures occur more commonly in carbonate formations, mainly due to their greater susceptibility to dissolution reactions, their architecture and their mechanical properties, which result in brittle behavior;
Geological age and depositional history: the older and deeper the formation, the less porous and permeable it tends to be. For this situation, when there is a concentration of stresses in the porous medium, the occurrence of fracturing is more susceptible;
Tectonic efforts: reservoirs that have already been found, in geological time, in regions of greater tectonic action are more prone to the occurrence of ductile deformations (such as folds) or brittle deformations (with faults, fractures and joints);
Types of fractures: while “fracture” is a generic term for discontinuities, it also denotes those in which no relative movement is observed between the blocks separated by the discontinuity. When there is relative movement between the faces of the discontinuity, due to a tensile force, it is a joint; if the force is shear, then it is a fault (Zerfass and Chemale Jr., 2011). In this description, fractures are understood as their most generic denotation;
The contribution of the fracture opening in the flow: Depending on the type of fracture and diagenetic changes, such as compaction, cementation or dissolution, fractures can be found in the subsurface, open or closed, i.e. they can function as preferential paths or as barriers to the fluid flow in the reservoir, respectively.

In order for the impact of fractures on fluid flow in the reservoir to be effectively considered during field development, they must be represented in the flow simulation model. To this end, it is important that the fractured environment is properly characterized, from its description based on the geological events that led to its formation, to the behavior of the fluids inside it during the exploitation of the reservoir, which can be modeled both numerically and experimentally in the laboratory, the latter being the object of this description.

### II—Classification of Naturally Fractured Reservoirs

Naturally fractured reservoirs can be classified based on the contribution of the rock matrix and fractures to the storage and fluid flow in the reservoir, considering their petrophysical properties (porosity and permeability). Nelson (2001) presents four main types of naturally fractured reservoirs:
Type (1)—impermeable matrix, where natural fractures guarantee the reservoir's porosity and permeability;
Type (2)—the matrix contributes with more than a half of the reservoir porosity (storage capacity), but does not contribute significantly to permeability and productivity;
Type (3)—the reservoir is already productive, that is, the matrix is porous and permeable, and the presence of fractures increases the productivity of the environment;
Type (4)—the reservoir contains fractures, which do not contribute to the porosity, nor to the permeability of the porous medium, acting as barriers to the flow.

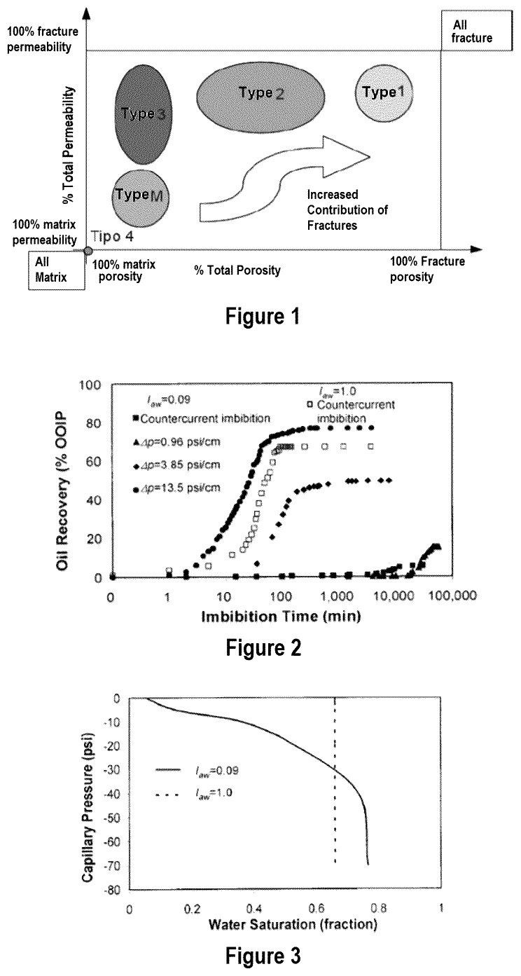

Nelson's (2001) classification is illustrated in FIG. 1, which is an illustrative diagram of the fracture contribution, in terms of porosity and permeability of the total reservoir, compared with the contribution of the rock matrix. A fifth type is included herein, the Type (M) reservoir, where fractures can increase the permeability of the already high porosity and permeability medium. However, Type (M) reservoirs typically function as barriers to flow.

For each type of naturally fractured reservoir represented in FIG. 1, Table 1 below describes the main characteristics related to hydrocarbon recovery (Baker and Kuppe, 2000).

TABLE 1
Type 1:
It is necessary to have a high intensity of fractures or high fracture
porosity in order to have an economically productive reservoir;
It can result in recent breakthroughs, as it is governed by the height and
vertical connectivity of the fractures;
Water influx is often followed by a rapid decline in oil production;
Fractures can promote the production of non-productive rocks;
Determining fracture porosity is critical for estimating recovery.
Type 2:
The efficiency of primary and secondary recovery is highly dependent on
how much the matrix is exposed to the fracture system;
Possibly the reservoir will suffer breakthrough early and a rapid decline in
production;
Development models must consider heterogeneities, for example, the
communication between matrix and fracture may vary areally;
The intensity of the fractures and their dip need to be known before
development begins;
Fractures increase productivity in reservoirs with low deliverability, i.e.,
the ability to promote flow;
Determining the permeability and heterogeneity of fractures is critical in
obtaining effective parameters and recovery potential.
Type 3:
Unusual responses to secondary recovery may occur;
The drainage area can be elliptical;
It may be difficult to recognize or detect the fracture system, as the
reservoir is already productive;
Fractures can then increase already economically viable opportunities;
As in the Type 2 reservoir, determining the permeability and heterogeneity
of the reservoir is a critical point.
Type 4:
Recovery is low due to severe compartmentalization of the reservoir;
If properly planned, field development can be optimized;
May present low secondary recovery due to compartmentalization.

Hydrocarbon recovery in naturally fractured reservoirs depends mainly on the magnitude of fracture permeability and how it varies spatially within the reservoir, in addition to the communication between matrix and fracture (represented in reservoir simulation by the transfer function). Good communication between matrix and fracture is essential for the productivity of the fractured reservoir, contributing to the achievement of high recovery factors. This communication between the media depends on the characteristics of both the fractures (aperture, permeability, spacing and direction of the fractures) and the matrix (porosity and permeability).

Thus, the complexity of representing the contribution of fractures to the production of the fractured reservoir in the numerical flow simulation can be seen, depending on the characterization of the geological structure and its impact on the recovery of hydrocarbons from the fractured medium, which methods are described below.

### III—Recovery Processes in Naturally Fractured Reservoirs

Every oil reservoir presents, in its initial condition, prior to production, a primary energy resulting from the characteristics of the rock and the fluids present therein, as well as the geometry and dimensions of the reservoir and its initial pressure. Associated with such characteristics and primary energy, primary recovery mechanisms can be indicated. However, the exploitation resulting from the action of such mechanisms makes it possible to produce a small part of the original volume of HC contained in the reservoirs. Therefore, it is common practice to supplement the reservoir's energy with the use of supplementary recovery mechanisms, which can be of two types, conventional and improved, described below. Attention is drawn to the specific issues of fractured reservoirs, with their particularities due to the discontinuity of the porous medium, as well as the hydraulic and capillary conductivity characteristics of the fractures:

### III.1. Primary Recovery

Primary recovery is closely related to the compressibility of the fluids and the formation. In the presence of free gas, the compressibility of connate and formation water can be disregarded. On the other hand, in under-saturated reservoirs, that is, where the reservoir pressure is higher than the oil saturation pressure, the latter must be taken into account, as it may influence recovery.

### III.1.1. Gas in Solution (or Volumetric Reservoir)

This production mechanism begins when the internal pressure of the reservoir decreases and reaches the bubble point of the fluid. This occurs first in the highest regions of the reservoir, where pressures are lower, and in the vicinity of producing wells. Initially, gas bubbles are formed within the rock matrix and, without forming a continuous phase, expand and contribute to the repressurization of the reservoir. When the gas bubbles reach critical gas saturation, they form a mobile phase, which is moved to the producing well. Firoozabadi (2000) draws attention to the importance of knowing this critical gas saturation to estimate the recovery of reservoirs under the influence of this process. Bourbiaux (2010) mentions that in the literature this parameter varies from 2 to 27% and that, for fractured reservoirs, high values of this parameter result in high recovery rates. Also according to Bourbiaux (2010), this mechanism, in general, results in low recovery rates.

### III.1.2. Gas Cap Expansion

The expansion of the gas cap is conditioned by the displacement of gas from the cap towards the oil zone, as a result of the depressurization of the reservoir. The effectiveness of this mechanism in production depends on the size of the layer, the volume of which must be greater than that of the oil zone itself.

### III.1.3. Water Influx

Similar to the gas cap mechanism, the influx mechanism involves the displacement of water from the aquifer underlying the reservoir towards the oil zone. This process occurs as the reduction in pressure at the oil-water contact propagates through the aquifer. The effectiveness of this mechanism depends on the relative size of the aquifer in relation to the oil zone, and can result in the highest levels of recovery among natural mechanisms.

### III.1.4. Combined Mechanism

The combined mechanism occurs in oil reservoirs that rely on the action of the gas cap and the aquifer simultaneously. In this mechanism, production flows and the depth of the perforations must be controlled in order to avoid water and gas conification. The formation of such cones is a function of the difference in viscosity of the fluids and the anisotropy of permeability of the reservoir rock, including its fractures.

### III.1.5. Gravitational Segregation

Related to the hydrostatic equilibrium between the fluid columns in the matrix and the fracture. After the formation of cones in the producing well and the disorderly distribution of fluids in the reservoir resulting from production, the closure of the producing well allows the natural reorganization of fluids in the reservoir according to density and gravity. By slowly reopening the wells, productivity is positively affected.

As cited by Legrand et al. (2011) depending on the structure and type of the fractured reservoir, more than one production mechanism can act in oil recovery. However, the gas-in-solution mechanism in fractured reservoirs generally does not promote significant oil recovery values in wells completed at the top of the reservoir. This is because, as soon as the gas reaches its critical saturation, it becomes mobile and moves to the highest parts of the reservoir, causing an increase in gas production combined with a rapid drop in reservoir pressure, since the reservoir's own energy (gas in solution) is being produced. However, this effect can be mitigated with enhanced supplemental recovery methods (also called EOR-Enhanced Oil Recovery), with the injection of additives that promote the reduction of the mobility ratio, increasing the recovery factor.

### III.2. Conventional Supplemental Recovery III.2.1.

### Water Injection

Water injection is the most widely used supplementary recovery process worldwide and has proven to be very efficient in some fractured reservoirs. The three main mechanisms acting on the displacement of oil by injected water are the spontaneous capillary soaking of the wettable rock matrix preferentially by water, the viscous displacement due to the pressure gradient generated by the flow in the fractures and the gravitational effect due to the difference in density between water and oil.

Attention in water injection is focused on the wettability of the fractured medium in question. For wettable media preferably with water and with high fracture intensity, due to capillary force, water will invade the matrix in a spontaneous soaking process and promote the concurrent and countercurrent displacement of the oil that was stored and, consequently, the sweeping efficiency will be greater, resulting in a better oil recovery. For wettability media ranging from intermediate to oil-wettable, recovery efficiency will not depend on spontaneous soaking of the matrix, but rather on other mechanisms such as forced soaking, viscous displacement and the gravitational effect.

Firoozabadi (2000) explains that although laboratory tests indicate low recovery by soaking for some media, the response shown in the field can be controversial. This is because the laboratory test consists of immersing a sample saturated with oil in water, forcing the soaking to occur in the opposite direction to the oil flow. Therefore, for non-wettable media preferentially to water, this test estimates low recovery rates. However, in a real situation, the injection of water into the field promotes soaking contrary to and/or concurrent with the oil flow, so that the increase in oil recovery is the result of the increase in the acting capillary pressure (suction), as illustrated in FIGS. 2 and 3 (Firoozabadi, 2000). FIG. 2 represents the oil recovery for water-wet reservoirs (curves with water-wetting identifier Iaw=1.0) and not wettable to water (Iaw=0.09)—the latter presents greater oil recovery. FIG. 3 represents the increase in suction (capillary pressure) for non-water-wettable (Iaw=0.09), while it remains constant for those wettable to water.

Examples include the Ekofisk field in the North Sea and the Midale field in Canada, which, although not being preferentially wettable with water, have shown good results with water injection. On the other hand, after water injection into the reservoir, a large amount of oil may remain either trapped in the matrix pores or in unswept regions of the reservoir. In these cases, the application of an advanced supplementary recovery process can be studied, as in the Midale field where miscible CO₂ injection was introduced, obtaining good recovery estimates.

Fractured gas reservoirs with connected aquifers are also subject to the water displacement mechanism. Significant quantities of gas are produced from low permeability carbonate reservoirs or fractured shales, and sometimes these reservoirs are overtaken by breakthroughs anticipated due to fractures that connect with active aquifers. The foothills of Alberta and northeastern British Columbia have reservoirs of this type. Initially, these reservoirs produce through the gas expansion mechanism and, after this period, when the adjacent aquifer starts to act, the soaking process occurs, which is generally quite slow. Lemonnier and Bourbiaux (2010), upon studying samples from Alberta reservoirs, concluded that, in some cases, the best production strategy would be to produce these reservoirs at the highest possible production rate until the wells began producing water, and then to use gravitational segregation by closing the wells, allowing the gas to “reorganize” in the porous medium. In fact, there are few published works on soaking in gas/water systems.

### III.2.2 Gas Injection

Similar to water injection, gas injection can be a conventional supplementary recovery method in which the injected gas has the function of replacing the produced fluid, repressurizing the reservoir, and displacing the oil, thus contributing to sweep efficiency and the recovery factor. The necessary condition for this behavior is the immiscibility between the injected fluids and the one originally contained in the reservoir.

As in a gas-liquid system the liquid phase will always be the wetting phase of the rock, the capillary force in this case acts to retain the oil inside the matrix. On the other hand, the magnitude of the capillary force is closely related to the reservoir pressure, becoming zero near the gas/oil miscibility pressure. The effect of viscous flow in fractures is less important than in water injection, due to the low viscosity of the gas, and is generally disregarded, except in cases of poorly fractured reservoirs.

The injected gas can come from an external source or from the reservoir itself, which may be the case for CO₂ and Nitrogen injection, in which the gas produced is reinjected.

### III.3. Advanced Supplemental Recovery

### III.3.1. Injection of Miscible Fluids

Gas injection into reservoirs is most often a compositional recovery process, since the injected gas will rarely be in full equilibrium with the in situ oil. Depending on the composition of both the injected gas and the oil and the thermodynamic conditions in the reservoir, miscibility of the two fluids can be achieved. However, these ideal displacement conditions in fractured reservoirs require the injected gas to penetrate the matrix due to gravitational forces and molecular diffusion between the two phases.

Two mechanisms can affect the efficiency of this type of recovery: reinfiltration and capillary continuity. Reinfiltration can direct oil flow into the low permeability matrix rather than direct oil flow into the high permeability fractures. Capillary continuity in the matrix can significantly increase final recovery. However, as per presented by Firoozabadi (2000), due to the difference in capillary pressure between matrix and fracture, the drainage rate in the fractured medium may be lower than in homogeneous matrices with low permeability.

It is worth noting that a conventional porous media flow simulator, the so-called black oil, does not allow the correct representation of the injection of miscible gas into reservoirs. In this case, so-called compositional simulators must be used.

### IV. Reservoir Characterization, Modeling and Simulation

A simulation model that best represents the behavior of fluids in the porous medium allows, based on the adjustment of the production history (such as records of the volumes of fluids produced and bottom-of-well pressures), a better predictability of its behavior. The more reliable the model, the better the reservoir management will be, resulting in its conscious, responsible and safe development.

A robust simulation model is based on a good representation of the reservoir rock, the fluids present therein and the interaction between these elements. In the case of naturally fractured reservoirs, this is the multiphase flow that occurs both in the matrix and in the fractures and even between both, and depends on properties such as capillary pressure and relative permeability, in addition to the transfer function.

One way of obtaining the parameters necessary for the representation in the reservoir numerical simulation of the fluids behavior in a naturally fractured environment is through laboratory tests, where one seeks to imitate, in the laboratory, the behavior that should occur in the subsurface, but under controlled conditions and on a much smaller scale.

### IV.1. Representation of the Naturally Fractured Environment

Due to its complexity, it requires a multidisciplinary study based on the integration of the geoscientists work and reservoir engineers. The former use information such as seismic data, reservoirs or similar outcrops, as well as the description of core samples and analyses of well profiles, in order to obtain parameters that describe the fractured medium, such as properties of the matrix (porosity and permeability) and fractures (direction and dip, frequency and opening, in addition to permeability). These parameters, among others, are used to construct the geological model, which is the basis for the simulation model, which must be worked on by engineers in order to represent the formation or production test records.

### IV.2. Interaction Between Rock and Fluids

Oil reservoirs contain three fluids in the porous medium: water, gas and oil. Once a pressure differential resulting from the opening of a producing well is imposed, these fluids move towards the lower pressure, competing with each other for space. The rock-fluid interaction properties that dictate this behavior are saturation, capillary pressure, and relative permeability. Such properties can be obtained in the laboratory, using rock samples (matrix and fractures that represent the fractured environment) and fluids similar to those contained in the reservoir.

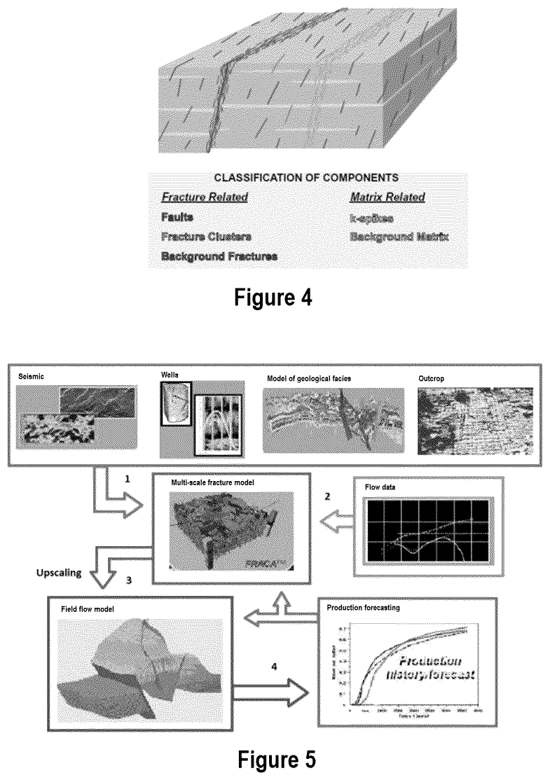

Phelps and Strauss (2002) relate the main constituent components of naturally fractured reservoirs and classify them as fault-related and matrix-related. Those related to the fault are the faults themselves, the fracture corridors and the diffuse fractures, that is, small-scale fractures that can also be called background fractures. As for those related to the matrix, there is the matrix itself and the super-permeability layers. These components are illustrated in FIG. 4.

The study of the development of naturally fractured reservoirs follows four fundamental steps for the description, modeling and simulation of the environment, as described below:
i. Construction of a geological model limiting the fracture system based on analysis, interpolation and extrapolation of information obtained from wells and derived from seismic data, sometimes complemented by outcrop analysis;
ii. Characterization of the fluid dynamic properties of this natural fracture system from flow data;
iii. Selection of a flow simulation model that adapts to the behavior presented by fractures and faults at various scales, as well as transfer of parameters from the geological fracture model with its conductive properties already calibrated to this new flow simulation model, at field scale;
iv. Simulation of reservoir productivity and recovery based on a physical assessment of the predominant flow mechanisms in the transfer between the two media.

These steps are correlated in FIG. 5 below. This methodology for analyzing naturally fractured reservoirs was proposed by Cascas et al. (2001) and summarized by Bourbiaux et al. (2005). The authors suggest a detailed geological analysis of the natural fracture information available in well data, such as well cores and image logs, subsurface seismic data, and outcrop data analogous to the rock under study. Although geologists are able to provide a detailed classification of the fracture systems existing in the environment, reservoir engineers are led to consider only the most relevant fracture families, those that generate the greatest impact on the flow. That is, in practice, when it comes to multi-scale fracture modeling, only two categories are considered: large-scale fractures (seismic and subseismic scale) and small-scale fractures (or diffuse fractures, observed in cores and image profiles).

### IV.3. Large Scale Failures

3D seismic data are the most reliable sources for building a fracture model. First, it is necessary to locate the position of the faults, identified as deterministic objects. Then, the fault system is extrapolated to the subseismic scale through a stochastic modeling process that honors the statistical parameters of the deterministic fault map (orientation, length, among others) and the fractal properties of the distribution of faults in space. The next constraints of this model are the maps of the presence of subseismic faults constructed based on the analysis of seismic morphology and various 3D seismic attributes. Large-scale fractures are considered to be the so-called fracture corridors (or fracture swarms), which are small aligned and grouped fractures, promoting a large-scale localized discontinuity.

### IV.4. Small Scale Fractures

Firstly, work is carried out to analyze and identify the families of fractures observed in the testimonies and image profiles. Different families are defined by average orientations, which are the result of structural characteristics and/or tectonic events. The nature of the geological facies also constitutes another classification parameter, because the expression of the fracturing event in a given facies is closely related to its mechanical properties. Once the fracture families and relevant facies have been identified, a fracture geomodel, describing the distribution of fracture parameters (orientation and density) for each family and each facies, can be constructed using a matrix geomodel. Finally, a stochastic model of the small-scale (diffuse) fracture systems can be generated for any position within the reservoir based on the parameters of this fracture geomodel.

Based on these analyses, a multiscale fracture model is constructed using stochastic methods limited by deterministic observations and genesis rules.

The construction of this fracture model has recently been carried out using discrete fracture network modeling software, also known by its acronym in English DFN—Discrete Fracture Network. For illustrative purposes, FIG. 6 presents the steps available in the FRACA™ software for constructing the DFN model. This method describes the fractures explicitly through finite elements, and allows a more realistic modeling of the fracture system, encompassing both the diffuse fracture geomodel and the subseismic fault model. These models are generally generated for a section of the reservoir and, through them, it is possible to simulate the fluid flow only in the fractures and, thus, obtain the fracture parameters necessary for field-scale simulation.

Another alternative methodology deals with discrete fracture and matrix modeling, with the acronym in English DFM—Discrete Fracture and Matrix. In this case, both the fractures and the rock matrix are discretized and it is possible to directly perform the flow simulation in a fractured medium. However, this method has certain particularities/limitations that do not currently allow its application on a field scale.

The importance of carrying out increasingly realistic characterization and modeling reflects the objective of providing more precise equivalent structural and hydrodynamic parameters for the simulation step and, thus, estimating, with the greatest possible accuracy, the future behavior of oil field production. Although the DFN model is capable of providing this more realistic modeling of the fractured medium, it cannot be directly used for field-scale flow simulation using current technologies. Therefore, even with limitations, the traditional dual-porosity, dual-permeability approach is used for field-scale simulation of flow in naturally fractured reservoirs. Therefore, it is necessary to perform a conversion (upscaling) of DFN model parameters to a predefined grid of a dual-porosity model. The main parameters required for the simulation of naturally fractured reservoirs are:
Porosity of the matrix and fracture;
Permeability of the matrix and fracture;
Matrix-fracture transfer parameter: characterized by a shape factor given by the dimensions of the matrix block, i.e., by the spacing of the fractures.

It is worth highlighting the large number of uncertainties that guide studies of naturally fractured reservoirs and the simplifications imposed throughout the process. Therefore, after constructing a fracture model, it is necessary to validate this model and calibrate its hydraulic characteristics, in order to provide adequate equivalent parameters for the flow simulation step.

### V—Evidence of a Naturally Fractured Reservoir

In the search to identify the impact of the presence of fractures in the reservoir, some indications, flow information in the field, are considered and correspond to a “dynamic” expression of the fractures. These indications, described below, come from both drilling information, flow behavior near wells or between wells, and historical production data from the field. However, it is worth highlighting that these indicators alone are not sufficient to describe the fracture system, and thus, they need to be compared with information from geology.

### Drilling Information:

A.1. Mud loss data: due to the presence of conductive fractures at a given depth in the well, it is possible to determine the hydraulic width of the fracture by the parallel plate equation, considering the appropriate fluid rheology (Bourbiaux, 2010). Only wide fractures are determined, with a hydraulic width of around 0.2 mm.

A.2. Increased penetration rate: An increase in penetration rate may indicate drilling into a zone with levels of fracturing, combined with the recovery of poor cuttings samples.

A.3. Well flow behavior:

A.3.1. Well Tests: Transient flow tests can provide information on the contrast in fluid storage capacity between matrix and fracture in a dual-porosity reservoir, as well as on the exchange factor between these media. They can also indicate faults and their distances from the well and are good for identifying high permeability contrasts between layers, such as high permeability layers, also known as ‘super-k layers’, which can sometimes be treated as horizontal fractures within the reservoir.

A.3.2. Well productivity: valuable information when compared with other well data such as distribution of faults and fractures along the well, petrophysical data measured from cores and flow data such as production logs (PLT—production logging tool, flowmeters).

The Productivity Index (PI) of the wells can also be evaluated. However, it is worth highlighting that this data does not only reflect the permeability in the region around the well, but also indicates possible changes in the rock (assessed by the effect Skin such as damage or stimulation to the well). Thus, as a preliminary attempt to identify the impact of fractures on flow, one can analyze the normalized IP (IP divided by the completion length) correlating it with the density of fractures along the completed section or another possible indicator of fracture such as the distance to the nearest fault.

A.3.3. Production Profile: the production profile of the entire completed section of the well allows specifying the contribution of each face to the permeability test, which can be compared with the permeability of the faces derived from the cores, in order to obtain the contribution of the fractures to the permeability of the reservoir for each facies (or interval). These contributions can then be compared with the fracture densities measured in the samples or in the image profiles of the respective facies, in order to qualify the conductivity of the fracture set defined by the geologist.

### B. Field Production History:

Distribution maps of well productivity and/or injectivity and accumulated fluid production, pressure maps, evolution of contact between fluids over time, breakthrough times, trends in water cut and in the evolution of the gas/oil ratio, RGO, over time, are examples of information obtained from the production history of a field. Although there is a need to anticipate these impacts, as there is little data prior to field production, this historical data is very important for confirming or comparing estimates made.

For already developed fields, multiphase production data is very important for future production optimizations or re-development of the field itself.

For compartmentalized reservoirs, data on the evolution of pressure and fluid composition (PVT properties of the oil, salinity of the water) during production are of interest to assess possible communication between the reservoir compartments in the presence of conductive faults.

### Description of the Preferred Embodiment of the Invention

Some of the elements and concepts described below may be generally known in the prior art. Therefore, certain details may be omitted from the description that follows with the understanding that the person skilled in the art has prior knowledge to fill these gaps. For example, the specific types of devices used, or underlying physical principles may be omitted without detracting from the description of the present invention.

The present invention describes a laboratory system for testing rock samples with fractures subjected to single-phase flow, using visible fluid and equipment invisible to a microtomography machine, allowing (i) the quantification of parameters that represent the flow in a fractured medium in the flow simulator; as well as (ii) images that portray the distribution of fluids in the medium, in different injection steps. It should be noted that the success of the experiment depends on the use of a fractured rock sample, whether natural or artificial, the obtaining of which is not part of this description.

The technology can be fully applied in the area of reservoir management and modeling in order to improve both the predictability and historical adjustment of the behavior of the naturally fractured reservoir, in addition to optimizing its management. As a consequence, more robust production curves will be obtained by reducing the degree of uncertainty regarding fluid flow in naturally fractured media.

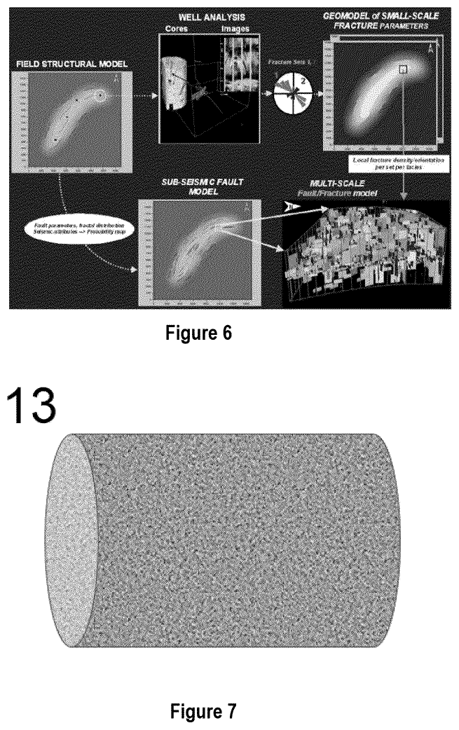

A porous plug like the one in FIG. 7 is artificially fractured, for example by laser, in order to obtain a fractured plug like the plug in FIG. 8. Each plug can have multiple fractures to study their influence on fluid flow through the plug.

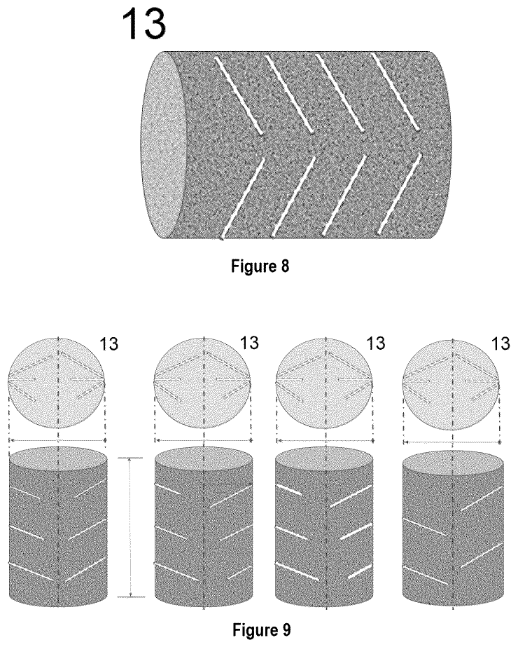

The fractures illustrated in the plug in FIG. 8 are not limiting. In fact, for a simulation closer to a real case, the fractures can be in different directions and depths in relation to the central axis of the plug. Fractures can be created in different arrangements of lengths, diameters, directions, angles and depths in relation to the central axis of the plug as in the example in FIG. 9.

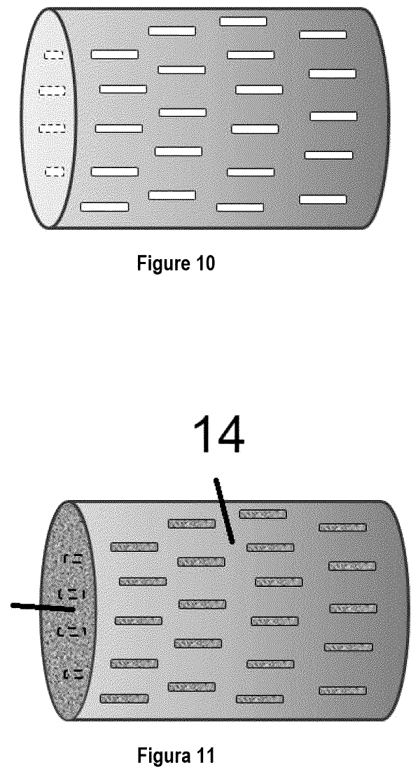

The fractured plug is surrounded by a Liner with holes as illustrated without limitation in FIG. 10. The liner is composed of a material machined from organic polymers, for example, Viton rubber, which is a synthetic rubber compound with fluoroelastomers in its composition. Preferably, the liner according to the present invention is a viton liner. The liner surrounding the plug is illustrated in FIG. 11.

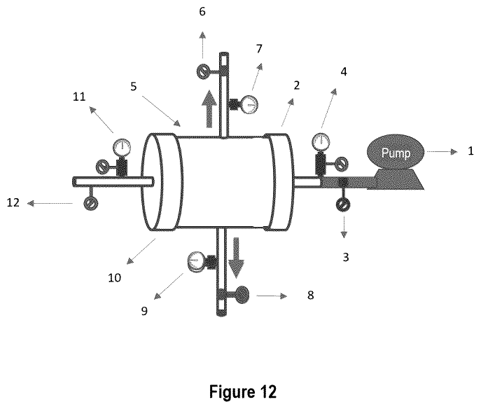

The fractured plug is positioned within a receptacle 5 specially designed to receive the plug 13 and allow the inlet and outlet of fluid through at least two, but preferably four channels. Each channel receives a duct for fluid flow, each fluid having a pressure gauge to measure the pressure of the fluid passing through the respective duct and a valve to allow or block the flow of the fluid through the respective fluid. At least one of the ducts is also coupled to a pump 1, for example, a peristaltic pump, for fluid pumping. The receptacle 5, ducts, pressure gauges, valves and pump form the flow measurement system according to the present invention, being illustrated without limitation in FIG. 12, where:
1—Pump to circulate fluid through the system;
2—Side cover for the watertight sealing of the receptacle;
3—Inlet valve for fluid flow access control;
4—Pressure gauge for assessing the pressure upstream of receptacle 5;
5—Receptacle to receive plug 13 (not shown);
6—Upper valve for air outlet when filling receptacle 5 with fluid;
7—Pressure gauge to assess the pressure in the upper part of receptacle 5;
8—Bottom valve for flow control through the bottom of receptacle 5;
9—Pressure gauge to assess the pressure in the lower part of receptacle 5;
10—Side cover for watertight closure of receptacle 5;
11—Pressure gauge for assessing the pressure downstream of receptacle 5;
12—Outlet valve for controlling horizontal fluid flow through receptacle 5;

According to the present invention, the fluid to be pumped into the receptacle 5 is a tracer fluid. Preferably, the tracer fluid is a non-ionic iodinated scintillating fluid.

According to the present invention, the receptacle 5 and the covers 2 and 10 must be made of material invisible to the microtomograph so as not to interfere with the analysis of the fluid flow. Such materials include materials made from organic polymers, for example, the plastic known as “PEEK”, which withstands extreme temperatures and high pressure.

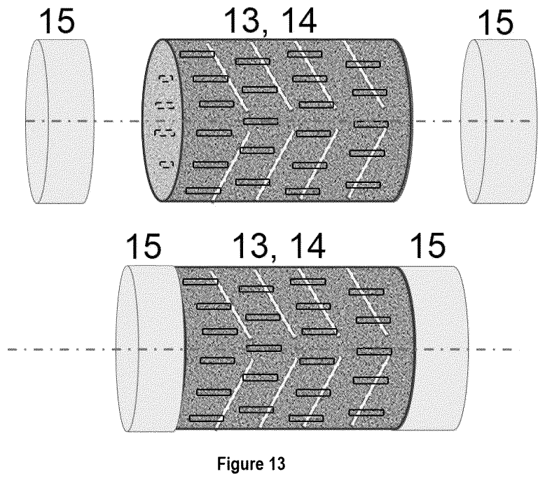

As seen in FIG. 13, adapters 15 can be inserted together with the plug 13 and liner 14 inside the receptacle 5 in order to improve their fit so that there is no relative movement between the plug 13 and liner 14 assembly in relation to the receptacle 5 during fluid flow. Adapters 15 are seen in more detail in FIG. 15.

Adapters 15 have an outer diameter that allows a fit in receptacle 5 and an inner diameter that allows a fit with liner 14 and plug 13. Thus, plug 13 is centered in relation to receptacle 5 and defining an annular space between receptacle 5 and plug 13. This annular space allows the fluid flow to be directed to the upper channel or the lower channel. The adapters 15 are also made of material that is not very sensitive to the microtomograph, for example, PEEK plastic.

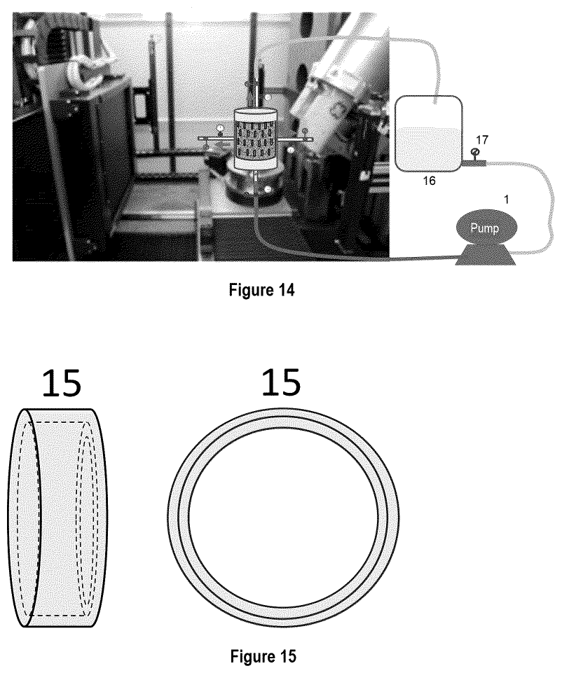

Once the invention system is positioned inside a microtomograph, as in FIG. 14. Pump 1 is connected in fluid communication with receptacle 5 and with a reservoir 16 containing tracer fluid regulated by an additional valve 17. Pump 1 is actuated to pump the tracer fluid through receptacle 5 and plug 13. The flow of tracer fluid through the plug may be directed to any one or a combination of the other outlets depending on the adjustment of valves 3, 6, 8 and 12.

The system of the invention makes it possible to evaluate the influence of fractures on the behavior of fluid flow within plug 13, by measuring the flow displaced to the fractures and, subsequently, to the annulus around plug 13 through the holes in liner 14. In the figures, these holes are represented by longitudinal cuts, however, the invention is not limited in this way.

The invention allows plugs to be tested with different amounts of fractures, from one to the maximum possible number of fractures that can be created, without weakening the structure of the plug. Fracture configurations can be varied to observe and measure the interference of the quantity and type of fractures on the flow through plug 13.

Experiments with the system of the invention are carried out inside a microtomograph so that it is possible to visualize the flow of the tracer fluid through the fractures and holes in the liner 14, providing monitoring of the behavior of the fluid flow inside the plug 13.

This entire procedure is carried out with the microtomograph activated to record the fluid flow. The data obtained in this way will allow the creation of more precise mathematical models of the flow of underwater fluids and muds in soils and fractured layers. This advancement will also allow the creation of more accurate simulations, aiding and improving the planning of future submarine operations.

A method of using the system described by the present invention is also provided, comprising the steps of:
Positioning the system inside a microtomograph;
Actuating the microtomograph; and
Actuating pump 1.

Although the aspects of the present disclosure may be susceptible to various modifications and alternative forms, specific embodiments have been shown by way of example in the drawings and have been described in detail herein. But it should be understood that the invention is not intended to be limited to the particular forms disclosed. Instead, the invention must cover all modifications, equivalents and alternatives that fall within the scope of the invention as defined by the following appended claims.

### REFERENCES

Zerfass H., Chemale jr. F. 2011. Geologia estrutural em ambiente rúptil: fundamentos físicos, mecânica de fraturas e sistemas de falhas naturais. Terræ Didatica, 7 (1): 75-85 http://www.ige.unicamp.br/terraedidatica/
NELSON, R. A.; Geological Analysis of Naturally Fractured Reservoirs. Gulf Publishing Co. Houston, 2001.
BAKER, R. O.; KUPPE, F.—“Reservoir Characterization for Naturally Fractured Reservoirs”—SPE 63286. 2000 SPE Annual Technical Conference and Exhibition. Dallas, Texas, 2000.
BOURBIAUX, B. Fractured Reservoir Simulation: A Challenging and Rewarding Issue. Oil Gas Science and Technology—Rev. IFP, Vol. 65, No. 2. 2010.
LEGRAND, N.; KOK, J.; NEFF, P.; CLEMENS, T. Recovery Mechanisms and Oil Recovery from a Tight, Fractured Basement Reservoir, Yemen. SPE 133086. SPE Reservoir Evaluation & Engineering, p. 493-504. August 2011.
FIROOZABADI, A.—“Recovery mechanisms in fractured reservoirs and field performance”—Journal Canadian Petroleum Tecnology, v. 39, n. 11, 2000
LEMONNIER, P.; BOURBIAUX, B. Simulation of naturally fractured reservoirs; State of art—Part 1; Physical mechanisms and simulator formulation. Oil & Gas Science and Technology, p. 239-262, 2010. Rueil-Malmaison.
PHELPS, R. E., STRAUSS, J., Capturing reservoir behavior by simulating vertical fracture and super-K zones in the Ghawar field. Society of Petroleum Engineers, SPE-79048-MS, SPE Reservoir Evaluation & Engineering, V.5, n.4, 2002.
CASCAS, M. C.; DANIEL, J. M.; LETOUZEY, J. Nested Geological Modelling of Naturally Fractured Reservoirs. Petroleum Geoscience, 7, p. S43-S52, 2001.
BOURBIAUX, B.; BASQUET, R.; LANGE, A.; HU, L. Y. Fractured Reservoir Management—Recent Progress and Promising Perspectives. 18th World Petroleum Congress. Block1, Forum 3 paper. Johannesburg, South Africa, 2005.

1. System for determining fluid flow with tracer in fractured rock sample, comprising:
a porous plug enclosed in a liner comprising holes;
a receptacle to receive the plug inside;
side covers for watertight closure of the receptacle;
a reservoir containing tracer fluid;
a pump in fluid communication with the receptacle and the reservoir;
a plurality of ducts connected to the receptacle, the pump and the reservoir for flow of the tracer fluid;
a plurality of valves, each valve installed in one of the ducts to control the flow of the tracer fluid;
a plurality of pressure gauges, each pressure gauge installed in one of the ducts to measure the pressure of the tracer fluid flowing in the duct.

2. System, according to claim 1, wherein the tracer fluid is a non-ionic iodinated scintillating fluid.

3. System, according to claim 1, wherein the receptacle also receives two adapters to center the plug in relation to the receptacle, defining an annular space.

4. System, according to claim 1 wherein it is operated inside a microtomograph.

5. Method for determining fluid flow with tracer in fractured rock sample, wherein it comprises the steps of:
Positioning the system as defined in claim 1 inside a microtomograph;
Actuating the microtomograph; and
Actuating the pump.
